(19) United States
(12) Patent Application Publication
(10) Pub. No.: US 2015/0359522 A1
(43) Pub. Date: Dec. 17, 2015

(54) POINT OF CARE URINE TESTER AND METHOD

(71) Applicant: Palo Alto Research Center Incorporated, Palo Alto, CA (US)

(72) Inventors: Michael I. Recht, San Carlos, CA (US); Joerg Martini, San Francisco, CA (US); Abhishek Ramkumar, Mountain View, CA (US); Peter Kiesel, Palo Alto, CA (US); Ben Hsieh, Mountain View, CA (US); Eugene M. Chow, Fremont, CA (US)

(21) Appl. No.: 14/307,193

(22) Filed: Jun. 17, 2014

(51) Int. Cl. A61B 10/00; G01N 15/14; G01N 21/25; G01N 21/64; E03D 9/00; G01N 27/416

(52) U.S. Cl. CPC A61B 10/007; E03D 9/00; G01N 27/416; G01N 21/255; G01N 21/645; G01N 15/1434; G01N 2201/06113; G01N 2201/02

(57) ABSTRACT

A urine capturing arrangement is configured to receive urine from a user of a toilet, and a chamber is fluidically coupled to the capturing arrangement. A diverter is fluidically coupled between the capturing arrangement and the chamber. The diverter is configured to divert a volume of the received urine to the chamber. A detection unit is configured to sense for presence of a predetermined characteristic in the volume of the urine and to generate at least one electrical signal comprising information about the predetermined characteristic.

### TECHNICAL FIELD

This application relates generally to techniques for analyzing urine samples. The application also relates to components, devices, systems, and methods pertaining to such techniques.

### SUMMARY

Various embodiments of the application are directed to a system that includes a capturing arrangement configured to receive urine from a user. The capturing arrangement may be used in conjunction with a toilet, urinal, bladder catheter, or any other such device configured to capture urine from a user. The system includes a chamber fluidically coupled to the capturing arrangement. A diverter is fluidically coupled between the capturing arrangement and the chamber. The diverter is configured to divert a volume of the received urine to the chamber. For example, the volume may be a volume of urine captured in the initial stream of urine, captured in mid-stream, or captured in the finishing stream of urine. A detection unit is configured to sense for presence of a predetermined characteristic in the volume of the urine and to generate at least one electrical signal comprising information about the predetermined characteristic. The system may include a communication device, such as a wireless transceiver, which facilitates transmission of detection data to a remote system or device. According to various embodiments, the system is adapted for mounting near, in, or on a toilet or urinal. In some embodiments, the system may be mounted on a toilet seat, for example. In some embodiments, the chamber containing the urine is detachable from the system and configured for transport to a remote location for assessment by a remotely located detection unit.

In accordance with other embodiments, a method involves capturing a sample of urine within a chamber of a testing apparatus. The method also involves sensing for presence of a predetermined characteristic in the urine within the chamber, and generating at least one electrical signal comprising information about the predetermined characteristic. The method may further involve transmitting data about the urine to a remote location.

The above summary is not intended to describe each disclosed embodiment or every implementation of the present disclosure. The figures and the detailed description below more particularly exemplify illustrative embodiments.

### BRIEF DESCRIPTION OF THE DRAWINGS

FIG. 1 is a flow chart of a non-invasive point-of-care testing method involving capture and assessment of urine at a home, clinic, or care facility in accordance with various embodiments;

FIG. 2 is a flow chart of a non-invasive point-of-care testing method involving capture and assessment of urine at a home, clinic, or care facility in accordance with other embodiments;

FIG. 3 is a flow chart of a non-invasive point-of-care testing method involving capture of urine at a home, clinic, or care facility and transporting of a urine sample to a remote testing facility in accordance with various embodiments;

FIG. 4 illustrates an apparatus for capturing and testing urine in accordance with various embodiments;

FIG. 5 illustrates an apparatus for capturing and testing urine in accordance with other embodiments;

FIG. 6 illustrates an apparatus for capturing and testing urine in accordance with further embodiments;

FIG. 7A illustrates an apparatus for capturing urine in accordance with various embodiments;

FIG. 7B illustrates an apparatus for capturing and testing urine in accordance with various embodiments;

FIG. 8 illustrates a non-invasive point-of-care testing apparatus adapted for use at a toilet in accordance with various embodiments;

FIG. 9A is a top view of the toilet shown in FIG. 8, with a capturing arrangement shown in a retracted configuration in accordance with various embodiments;

FIG. 9B shows the capturing arrangement and extension arm of FIG. 9A in a deployed configuration;

FIG. 10A is a top view of the toilet with a capturing arrangement shown in a retracted configuration in accordance with various embodiments;

FIG. 10B shows the capturing arrangement and extension arm of FIG. 10A in a deployed position in accordance with various embodiments;

FIG. 10C is a top view of the toilet with a capturing arrangement shown in a retracted configuration in accordance with various embodiments;

FIG. 10D shows the capturing arrangement and extension arm of FIG. 10C in a deployed position in accordance with various embodiments;

FIG. 11 is a block diagram of a urine testing apparatus adapted for deployment at a toilet in accordance with various embodiments;

FIG. 12 shows an embodiment of a urine diverter configured to facilitate capture of bladder urine within a testing apparatus deployed at a toilet in accordance with various embodiments;

FIG. 13 shows an embodiment of a urine diverter configured to facilitate capture of bladder urine within a testing apparatus deployed at a toilet in accordance with other embodiments;

FIG. 14 shows an embodiment of a urine diverter configured to facilitate capture of bladder urine within a testing apparatus deployed at a toilet in accordance with further embodiments;

FIG. 15 is a diagram of a detection unit which includes a compact flow cytometer configured to perform single or multiple analyte detection performed on a urine sample acquired in real-time by a testing apparatus deployed at a toilet in accordance with further embodiments; and

FIG. 16 conceptually illustrates a device configuration useful for performing urinalysis according to some embodiments.

The figures are not necessarily to scale unless otherwise indicated. Like numbers used in the figures refer to like components. However, it will be understood that the use of a number to refer to a component in a given figure is not intended to limit the component in another figure labeled with the same number.

### DESCRIPTION

In the following description, reference is made to the accompanying set of drawings that form a part of the description hereof and in which are shown by way of illustration several specific embodiments. It is to be understood that other embodiments are contemplated and may be made without departing from the scope of the present disclosure. The following detailed description, therefore, is not to be taken in a limiting sense.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. The use of numerical ranges by endpoints includes all numbers within that range (e.g. 1 to 5 includes 1, 1.5, 2, 2.75, 3, 3.80, 4, and 5) and any range within that range.

Non-invasive point-of-care (POC) diagnostic instruments offer the unique capability of performing clinically-relevant measurements in biological fluids such as urine at the time of fluid sample extraction/excretion from the patient, without the need for sample storage and shipping required for lab-based tests. Non-invasive POC diagnostic instruments also enable fast turn-around-times providing timely results and feedback for titrating therapy. Analysis of urine, for example, is unique because of the timely occurrence or requirement of urinary excretion by the body, providing ample fluid volume to perform analytics to detect molecules or proteins of interest in the urine and assess the onset or progression of a disease state.

Kidney transplant patients, for example, are among many who could benefit immensely from a non-invasive diagnostic tool for urinalysis performed at the urine collection device, e.g., toilet, urinal, bladder catheter, etc., in order to monitor the health of the new kidney function in the body on a daily basis. There are close to 200,000 kidney transplant patients in the United States, and 20% of these patients are likely to have transplant failure in the first five years, with a post graft failure annual cost of $80,000/patient. Aside from invasive kidney biopsy, currently there is no gold-standard device for non-invasively monitoring or detecting the onset of a transplant failure in patients. A recent seminal urinalysis clinical trial demonstrated a positive correlation between increase in lymphocytes and renal tubular cells (RTCs) with kidney transplant rejection as early as five days prior to the event (see “Analysis of Urine Sediment for Cytology and Antigen Expression in Acute Renal Allograft Rejection An Alternative to Renal Biopsy,” Priti Chatterjee, MD, Sandeep R. Mathur, MD, Amit K. Dinda, MD, Sandeep Guleria, MS, Sandeep Mahajan, MD, V. K. Iyer, V. K. Arora, MD, Am J Clin Pathol. 2012; 137(5):816-824).

Although sample collection for urine cytology is simple, the analysis must still be performed in a clinical lab using existing techniques. A low cost, fully-automated point of care device for urine cytology consistent with embodiments of the present disclosure enables, at minimum, daily samples to be collected and analyzed on-site for cellular content. The more consistent frequency of sampling allows the cytological signs of allograft rejection to be identified as early as possible. More frequent sampling improves knowledge of urine cytology markers for rejection and potentially provides more advance warning of rejection so that proper treatment can be initiated.

Embodiments are directed to a non-invasive POC testing apparatus and method for use at a urine collection device, such as a toilet, urinal, bladder catheter and the like. In some of the examples provided herein, the urine collection device is referred to and shown as a toilet. It will be appreciated that the approaches described herein are also applicable to any other type of urine collection device, such as a urinal, a bladder catheter, etc. Capture and testing of a urine sample at the toilet (or other urine collection device) provides for assessment of the urine sample immediately after capture, thereby improving the quality of the assessment. The duration of time between collecting a urine sample and testing the sample can significantly impact the quality and accuracy of the assessment. The following changes, for example, occur in a urine sample with time after capture: 1) decreased clarity due to crystallization of solutes, 2) rising pH, 3) loss of ketone bodies, 4) loss of bilirubin, 5) dissolution of cells and casts, and 6) overgrowth of contaminating microorganisms. In general, urinalysis may not reflect the findings of fresh urine if the sample is greater than 1 hour old. Embodiments of the disclosure provide for testing (e.g., performing urinalysis) of a urine sample, such as a mid-stream sample, immediately after capture of the sample.

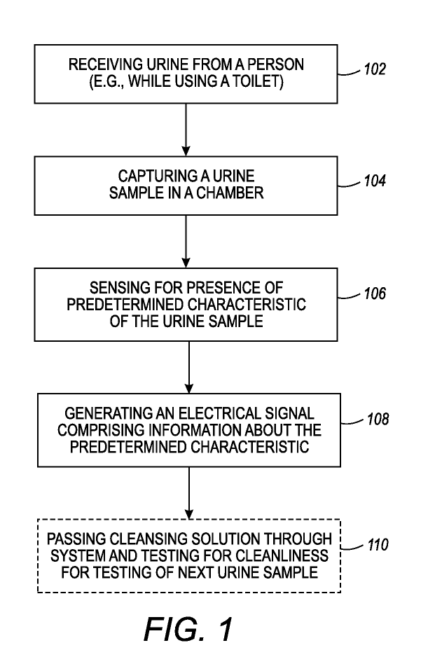

According to various embodiments, and with reference to FIG. 1, a non-invasive POC testing method involves receiving 102 urine, e.g., from a person using a toilet, urinal, bladder catheter, or other urine collection device, and capturing 104 a sample of the urine within a chamber of a testing apparatus at the urine collection device. The chamber may be, for example, a cup or other such device. The method also involves sensing for presence 106 of a predetermined characteristic in the volume of the urine captured, and generating 108 at least one electrical signal comprising information about the predetermined characteristic. The method may further involve passing 110 a cleansing solution through the testing apparatus and testing for cleanliness in advance of a subsequent urine test.

In some embodiments, each of the processes illustrated in FIG. 1 is performed at the urine collection device, e.g., toilet. In other embodiments, the receiving 102 and urine capturing 104 processes are performed at the urine collection device, and the sensing 106 and generating 108 processes are performed by a detection unit, which may be located near to (e.g., on a table in the bathroom) or remotely from (e.g., in a room near the bathroom or in a distant facility) the receiving and urine capturing apparatuses at the toilet. In some embodiments, each of the processes illustrated in FIG. 1 is performed with no or only minimal intervention by the person using the toilet. In other embodiments, the receiving 102 and capturing 104 processes are performed with no or only minimal intervention by the person using the toilet, and the sensing 106 and generating 108 processes involve transporting a chamber of the captured urine sample to a testing apparatus and initiating an analysis of the captured urine sample (e.g., such as by pushing a button on the testing apparatus). In some embodiments, the chamber is fluidically coupled, e.g., by a tube, pipe, or other fluid transporting element, to a detection unit that performs the sensing and generating. The captured urine sample is transported to the detection unit via the tube to the detection unit wherein the sensing and generating processes are implemented.

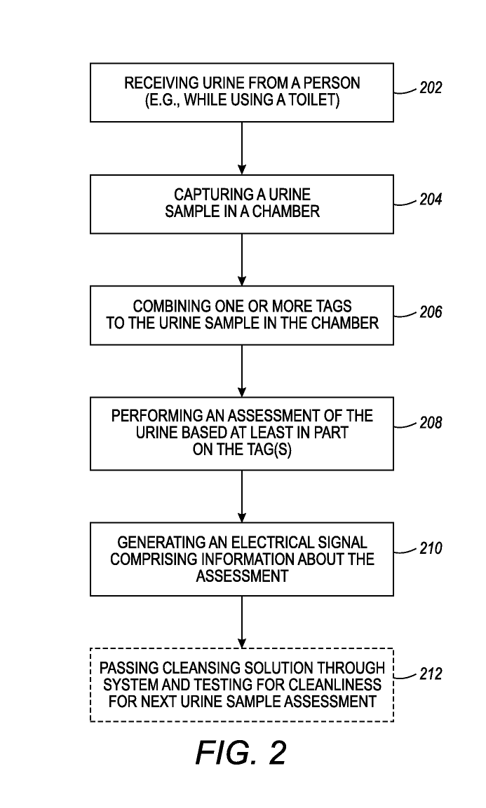

In accordance with some embodiments, and with reference to FIG. 2, a non-invasive POC testing method involves receiving 202 urine from a person, and capturing 204 a sample of the received urine within a chamber of a testing apparatus at the toilet. The method also involves combining 206 one or more specificity tags with the urine sample in the chamber. Each of the one or more specificity tags are selected to attach to a specified component, composition, substance, molecule, compound, chemical, biological structure, object or other constituent feature of the urine sample (collectively referred to herein as an analyte). Each of the one or more tags has a characteristic that is detectable (e.g., signature emission spectra), allowing for indirect detection of the analyte to which the tag attaches. The method further involves performing an assessment 208 of the urine based at least in part on detection of the tag(s), and generating 210 at least one electrical signal comprising information about the assessment (e.g., presence of an analyte(s), concentration of an analyte(s)). For example, in one scenario, a first specificity tag is configured to attach to a first analyte and a second specificity tag is configured to attach to a second analyte. Detection of the first specificity tag during the analysis indicates the presence and/or concentration of the first analyte and detection of the second specificity tag during the analysis indicates the presence and/or concentration of the second analyte. The method may further involve passing 212 a cleansing solution through the testing apparatus and testing for cleanliness in advance of a subsequent urine test.

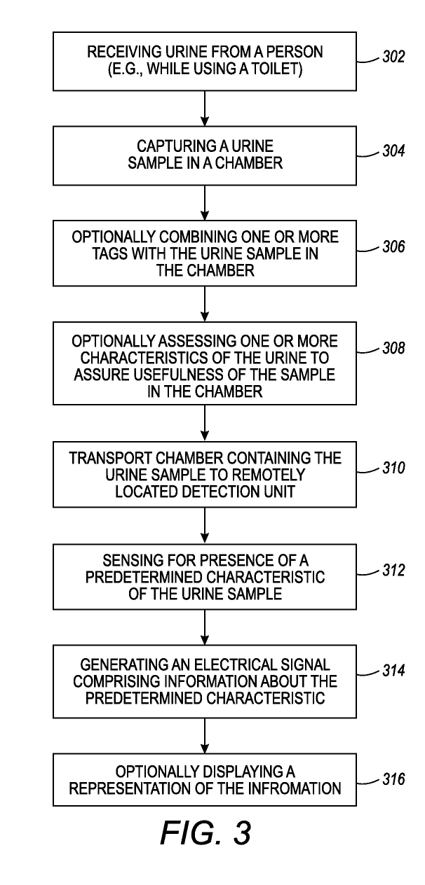

According to other embodiments, and with reference now to FIG. 3, a non-invasive POC testing method involves receiving 302 urine from a person using a toilet, and capturing 304 a sample of the received urine within a chamber of a testing apparatus at the toilet. The method may optionally involve combining 306 one or more tags with the urine sample in the chamber. The method may optionally involve assessing 308 one or more characteristics (e.g., color, cloudiness, concentration) of the urine as a screening measure to assess the usefulness of the urine sample captured in the chamber. This screening assessment 308 provides a general indication of whether or not the urine sample captured in the chamber will provide useful information when subjected to more sophisticated testing.

According to the embodiment shown in FIG. 3, the chamber containing the urine sample is removed from the urine capturing apparatus at the urine collection device, and transported 310 to a remotely located detection unit. The detection unit may be located in the same room as the toilet but spaced apart from the toilet (e.g., situated on table or shelf in the bathroom). The chamber, e.g., a cup, containing the urine sample may be transported from the urine collecting device to the detection unit by the person who produced the sample or by a caregiver. In some implementations, the chamber of the urine capturing arrangement may be fluidically connected to the detection unit, e.g., by a tube. The urine sample can be transported from the chamber of the urine capturing arrangement, which is located at the urine collection device, to the detection unit via the tube or pipe. In some scenarios, the system can include a pump configured to pump the urine sample from the chamber to the detection unit.

In another scenario, the detection unit may be located in another room (e.g., a lab) distant from the bathroom within the same building or complex. The detection unit may be located distantly from the bathroom (e.g., in another city or state) and the chamber containing the urine sample may be transported (e.g., shipped, mailed) to the distantly located detection unit, which may be operated by a lab technician, for example.

The methodology of FIG. 3 provides for a real-time capture of a urine sample in a chamber wherein the sample may also include one or more tags mixed with the urine sample after capture. The chamber may be detachable from the urine capturing arrangement and suitable for transport. Alternatively, the chamber may be fluidically connected to a detection unit that is located remotely from the chamber. The methodology of FIG. 3 can also provide for an initial screening of the urine sample within a brief time period after capture, thereby reducing delays and costs resulting from performing urinalysis on contaminated or unusable urine sample. The method of FIG. 3 further involves sensing for presence 312 of a predetermined characteristic in the volume of the urine within the chamber, and generating 314 at least one electrical signal comprising information about the predetermined characteristic. The method may also involve passing a cleansing solution through the apparatus at the toilet and testing for cleanliness in advance of a subsequent urine test.

In some embodiments, testing for cleanliness of the apparatus at the toilet involves sensing for an analyte of urine and determining whether a sense signal indicative of analyte presence exceeds a predetermined threshold. If not, the test indicates that the apparatus is sufficiently clear of urine from a previous test and is ready for another test cycle. If the test indicates presence of the analyte exceeds the predetermined threshold, a signal is generated indicating that the apparatus requires cleaning. Cleaning may also be indicated by other metrics, such as elapsed time since last cleaning, numbers of uses since last cleaning, signal characteristic changes (e.g. too high, too low, too noisy).

The signal includes information about one or more predetermined characteristics of the urine sample and can be transmitted from the apparatus at the urine collection device, e.g., toilet, to another system or device accessible to the person providing urine samples, a caregiver or a clinician. Optionally, a representation of the information carried by the signal may be displayed on a display. In some embodiments, the display may be located near the urine collection device, e.g., toilet, and may comprise one or more light emitting diodes (LEDs) that are activated or deactivated based on the information in the signal. For example, the display may comprise one red and one green LED, wherein activation of the green LED indicates a normal range of the predetermined characteristic of the urine and activation of the red LED indicates an abnormal range of the predetermined characteristic. In this configuration, the urine is analyzed and information about the urine characteristics are displayed to the user within a brief interval after capturing the urine.

In some embodiments, a more complex display may be used that is capable of providing a graphical or textual representation of the information. In some embodiments, the need for cleaning may be transmitted or indicated on the display. In some embodiments, an automatic cleaning process (e.g. flushing) and/or testing for cleanliness, may be activated automatically.

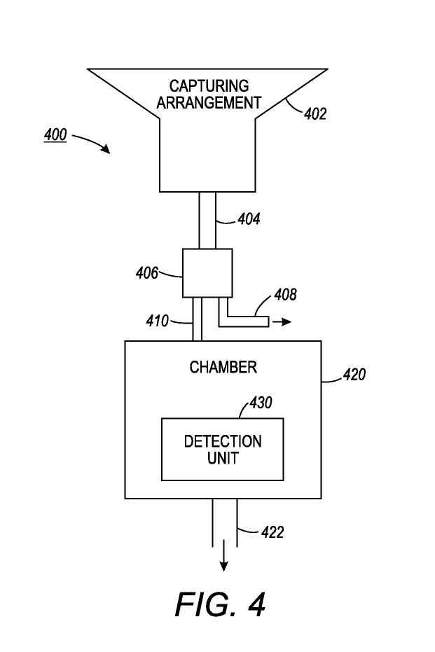

Turning now in FIG. 4, there is illustrated an apparatus 400 for capturing and testing urine in accordance with various embodiments. The apparatus 400 includes a urine capturing arrangement 402 which is configured to receive urine from a user of the toilet. The urine capturing arrangement 402 may include a funnel or other structure that facilitates capturing of urine from the user of a urine collection device, such as a toilet, urinal, bladder catheter, etc. The urine capturing arrangement 402 is fluidically coupled to a chamber 420. Coupled between the capturing arrangement 402 and chamber 420 is a diverter 406. The diverter 406 can be fluidically coupled to the capturing arrangement 402 via a conduit 404 and to the chamber 420 via conduit 410. The conduits 404, 408, and 410 may be flexible or rigid hollow members. The diverter 406 is configured to divert a volume of urine received by the capturing arrangement 402 to the chamber 420.

The diverter 406 includes a first port coupled to the conduit 410 which passes the volume of urine to be analyzed to the chamber 420 where the urine is contained. A second port of the diverter 406 is fluidically coupled to a conduit 408 which diverts urine received by the capturing arrangement 402 that is not part of the volume to be analyzed away from the chamber 420, e.g., into the toilet bowl. For example, in some scenarios, the second port (or a third port) of the diverter 406 can be used to divert excess urine away from the chamber 420 (e.g., into the toilet bowl) after a sufficient volume of urine has been captured in the chamber 420. In some embodiments that involve capturing mid-stream urine, the first- and last-voided urine may be diverted via the second port.

In the embodiment shown in FIG. 4, a detection unit 430 is situated at or near the chamber 420 and configured to sense for presence of a predetermined characteristic in the volume of the urine contained in the chamber 420. Depending on the complexity of the detection unit 430, a single characteristic or a multiplicity of characteristics of the urine may be subject to assessment by the detection unit 430. The detection unit 430 is configured to generate at least one electrical signal comprising information about the predetermined characteristic(s). In accordance with embodiments that provide for detection of a multiplicity of predetermined characteristics of the urine contained in the chamber 420, the detection unit 430 may be configured to generate a multiplicity of electrical signals each comprising information about one of the predetermined multiplicity of characteristics. After the detection unit 430 completes the assessment of the urine sample, the urine contained in the chamber 420 is expelled via an exit port 422. Prior to a subsequent urine capture and assessment cycle, a cleansing operation may be conducted. Part of the cleaning protocol may include passing of a cleansing solution through the testing apparatus 400 or parts of the testing apparatus so as to flush any remaining urine from the previous test cycle out of the apparatus 400.

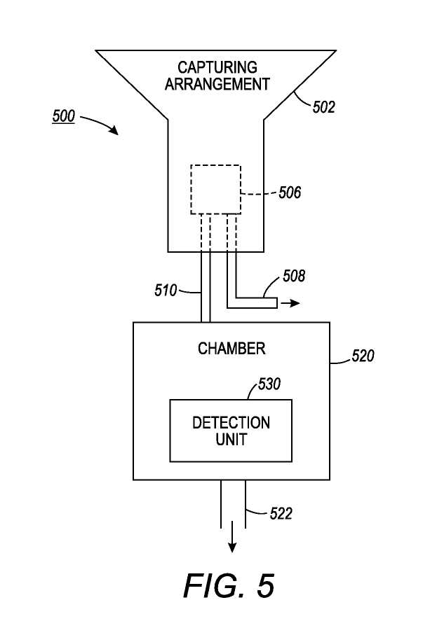

FIG. 5 shows an apparatus 500 for capturing and testing urine from a person in accordance with other embodiments. The apparatus 500 includes a urine capturing arrangement 502 which is configured to receive urine from a user. In the embodiment shown in FIG. 5, the capturing arrangement 502 incorporates a diverter 506 which is configured to divert a volume of urine to be tested that has been received by the capturing arrangement 502 to the chamber 520 for containment therein via conduit 510. A second port of the diverter 506 is fluidically coupled to a conduit 508 which diverts non-test urine received by the capturing arrangement 502 away from the chamber 520, such as into a toilet bowl. The second port (or a third port) of the diverter 560 can be used to divert excess urine away from the chamber 520 (e.g., into the toilet bowl) after a sufficient volume of urine to be tested has been captured and contained in the chamber 520.

A detection unit 530 is shown situated at or near the chamber 520 and configured to sense for presence of a predetermined characteristic in the volume of the urine contained in the chamber 520. A single characteristic or a multiplicity of characteristics of the volume of urine may be subject to assessment by the detection unit 530. The detection unit 530 is configured to generate at least one electrical signal comprising information about the predetermined characteristic. According to embodiments that provide for detection of a multiplicity of predetermined characteristics of the volume of urine contained in the chamber 520, the detection unit 530 is configured to generate a multiplicity of electrical signals each comprising information about one of the predetermined multiplicity of characteristics. After the detection unit 530 completes the assessment of the urine sample, the urine contained in the chamber 520 can be expelled via an exit port 522. Prior to a subsequent urine capturing and assessment cycle, a cleansing operation may be conducted in which the cleansing solution is passed through the testing apparatus 500, or parts of the testing apparatus, so as to flush any remaining urine from the previous test cycle out of the apparatus 500.

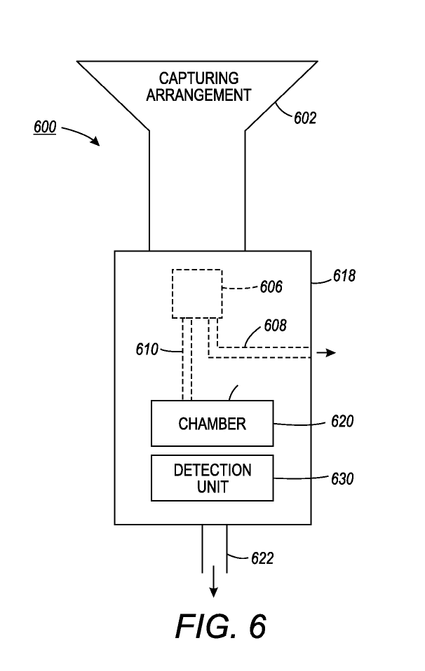

FIG. 6 illustrates an apparatus 600 for capturing and testing urine from a person in accordance with various embodiments. The apparatus 600 includes a capturing arrangement 602 which is configured to receive urine from a user. The capturing arrangement 602 is fluidically coupled to a chamber apparatus 618. In the embodiment shown in FIG. 6, the chamber apparatus 618 incorporates both a chamber 620 and a diverter 606 which is configured to divert a volume of urine, e.g., mid-stream urine, to be tested that has been received by the capturing arrangement 602 to the chamber 620 for capture therein via conduit 610. A second port of the diverter 606 is fluidically coupled to a conduit 608 which diverts non-test urine, e.g., first-voided urine, received by the capturing arrangement 602 away from the chamber 620, such as into the toilet bowl. The second port or a third port of the diverter 606 can be used to divert excess urine, e.g., last-voided urine, away from the chamber 620 (e.g., into the toilet bowl) after a sufficient volume of urine has been captured in the chamber 620.

A detection unit 630 is shown situated at or near the chamber 620 and configured to sense for presence of a predetermined characteristic in the volume of the urine contained in the chamber 620. A single characteristic or a multiplicity of characteristics of the volume of urine may be subject to assessment by the detection unit 630. The detection unit 630 is configured to generate at least one electrical signal comprising information about the predetermined characteristic. According to embodiments that provide for detection of a multiplicity of predetermined characteristics of the volume of urine contained in the chamber 620, the detection unit 630 is configured to generate a multiplicity of electrical signals, each comprising information about one of the predetermined multiplicity of characteristics. After the detection unit 630 completes the assessment of the urine sample, the urine contained in the chamber 620 is expelled via an exit port 622. Prior to a subsequent urine capture and assessment cycle, a cleansing operation may be conducted in which the cleansing solution is passed through the testing apparatus 600 or parts of the apparatus so as to flush any remaining urine from the previous test cycle out of the apparatus 600.

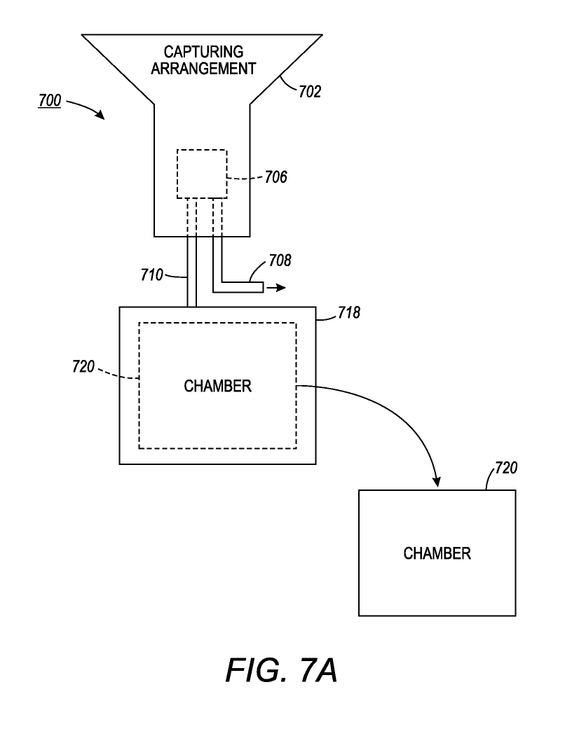

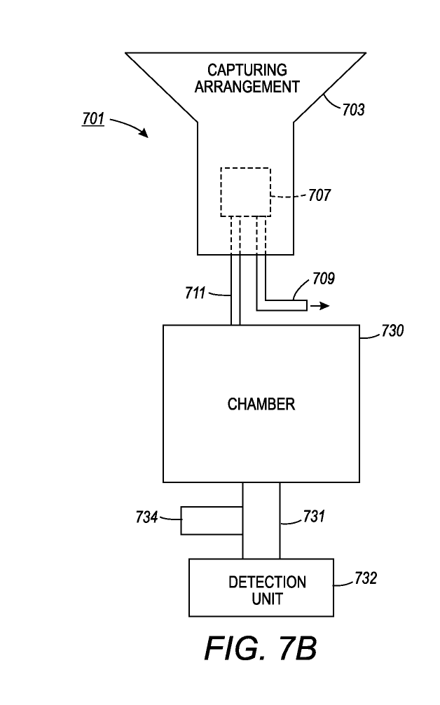

FIG. 7A shows an apparatus 700 for capturing and testing urine from a person in accordance with other embodiments. The apparatus 700 includes a capturing arrangement 702 which is configured to receive urine from a user. The apparatus 700 includes a capturing arrangement 702 which is fluidically coupled to a chamber apparatus 718. Fluidically coupled between the capturing arrangement 702 and chamber apparatus 718 is a diverter 706. The diverter 706 may be incorporated as an integral component of the capturing arrangement 702 or, alternatively, as a separate component fluidically coupled to the capturing arrangement 702. For purposes of explanation, the diverter 706 in the embodiment shown in FIG. 7A is integrated within the capturing arrangement 702. The chamber apparatus 718 includes a detachable chamber 720.

The diverter 706 is configured to divert a volume of urine to be tested that has been received by the capturing arrangement 702 to the detachable chamber 720 via conduit 710 for capture therein. A second port of the diverter 706 is fluidically coupled to a conduit 708 which diverts non-test urine received by the capturing arrangement 702 away from the chamber 720, such as into the toilet bowl. The second port or a third port of the diverter 706 can be used to divert excess urine away from the chamber 720 (e.g., into the toilet bowl) after a sufficient volume of urine has been collected in the chamber 720. After the detachable chamber 720 has collected a sufficient volume of urine, the chamber 720 can be removed from the chamber apparatus 718. The detachable chamber 720 incorporates a sealing arrangement that allows urine to be introduced into the chamber 720 and prevents urine from unintentionally escaping the chamber 720. The detachable chamber 720 may be transported to a detection unit configured to receive the chamber 720. The detection unit is configured to sense for presence of a predetermined characteristic in the volume of the urine and to generate at least one electrical signal comprising information about the predetermined characteristic.

Prior to a subsequent urine capture and assessment cycle, a cleansing operation may be conducted in which the cleansing solution is passed through the testing apparatus or parts of the apparatus so as to flush any remaining urine from the previous test cycle out of the apparatus. In some implementations, the container may be disposable. In other implementations, a portion of the container may be disposable or the container may be reusable. A reusable container or a reusable portion of the container may be cleaned during the cleaning cycle.

FIG. 7B shows an apparatus 701 for capturing and testing urine from a person in accordance with other embodiments. The apparatus 701 includes a capturing arrangement 703 which is configured to receive urine from a user. The capturing arrangement 703 is fluidically coupled to a chamber 730. Fluidically coupled between the capturing arrangement 703 and chamber 730 is a diverter 707. The diverter 707 may be incorporated as an integral component of the capturing arrangement 703 or, alternatively, as a separate component fluidically coupled to the capturing arrangement 703. For purposes of explanation, the diverter 707 in the embodiment shown in FIG. 7B is integrated within the capturing arrangement 703.

The diverter 707 is configured to divert a volume of urine to be tested that has been received by the capturing arrangement 703 to the chamber 730 via conduit 711 for capture therein. A second port of the diverter 707 is fluidically coupled to a conduit 709 which diverts non-test urine received by the capturing arrangement 703 away from the chamber 730, such as into the toilet bowl. The second port or a third port of the diverter 707 can be used to divert excess urine away from the chamber 730 (e.g., into the toilet bowl) after a sufficient volume of urine has been collected in the chamber 730.

The chamber 730 is fluidically connected to the detection unit 732 via a tube, pipe, or other device 731. The urine in the chamber 730 can be transported from the chamber 730 to the detection unit 732 through the pipe 731. Optionally, the transport of the volume urine to be tested between the chamber 730 and the detection unit 732 may be facilitated by a pump 733.

In some embodiments, the detection unit may be located in the same room as the capturing arrangement and chamber, e.g., on a table or counter in a bathroom. The detection unit is configured to sense for presence of a predetermined characteristic in the volume of the urine and to generate at least one electrical signal comprising information about the predetermined characteristic.

Prior to a subsequent urine capture and assessment cycle, a cleansing operation may be conducted in which the cleansing solution is passed through the testing apparatus or parts of the apparatus so as to flush any remaining urine from the previous test cycle out of the apparatus. In some implementations, the entire container may be disposable or a portion of the container may be disposable. In other implementations, the container may be reusable. A reusable container or a reusable portion of the container may be cleaned during the cleaning cycle.

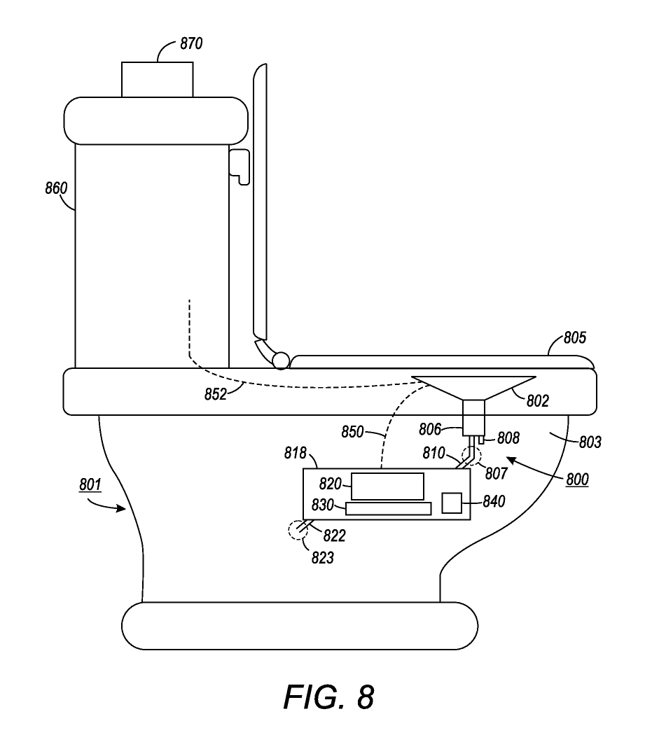

FIG. 8 illustrates a non-invasive point of care (POC) testing apparatus 800 adapted for use at a toilet in accordance with various embodiments. As shown in FIG. 8, the toilet 801 includes a tank 860, bowl 803, and seat 805. According to the embodiment shown in FIG. 8, a testing apparatus 800 includes components positioned within the bowl 803 of the toilet 801 and components positioned near or on an external surface of the toilet bowl 803. In some configurations, the components of the testing apparatus 800 may be coupled to the toilet seat 805. In this configuration, installation of the testing apparatus 800 for use with a toilet is simplified because installation can be accomplished by installing a toilet seat equipped with the testing apparatus 800.

Components of the testing apparatus 800 that are positioned within the toilet bowl 803 include a capturing arrangement 802 and a diverter 806 which can be integral to or separate from the capturing arrangement 802. In the example illustrated in FIG. 8, the diverter 806 is integrated within the capturing arrangement 802. The diverter 806 is fluidically coupled to a chamber 820 situated within a housing 818 mounted near or on an external surface of the toilet bowl 803. A conduit 810 may be routed between the toilet seat 805 and the toilet bowl 803 or may pass through an access hole 807 provided in the toilet bowl 803. If the conduit passes through the access hole 807, a seal is provided between the conduit 810 and the toilet bowl 803 at the access hole 807 to prevent toilet water from exiting the toilet bowl 803 via the access hole 807. The diverter is configured to pass a volume of urine received by the capturing arrangement 802 to the externally positioned chamber 820 via the conduit 810. Non-test, excess urine is passed through a conduit 808 and dispensed into the toilet bowl 803. Conduit 808 or another conduit (not shown) can be used to divert excess urine into the toilet bowl 803 after a sufficient volume of urine has been captured.

A detection unit 830 is situated within the housing 818 and in proximity to the chamber 820. The detection unit 830 is configured to sense for presence of the predetermined characteristic in the volume of the urine contained within the chamber 820. In some embodiments, examples of which are described herein, the detection unit 830 can include a compact, optical flow cytometer fluidically coupled to the chamber 820. The detection unit 830 is further configured to generate at least one electrical signal comprising information about the predetermined characteristic. In some embodiments, information generated by the detection unit 830 is stored in memory and may be periodically communicated to a remote system or device via a communication device 840. In some embodiments, the communication device 840 includes a wireless transceiver. The communication device 840 may be configured to implement a variety of wireless communication protocols, including those conforming to one or more of an IEEE 802.11b/g/n/ac/ad/af/ah, Bluetooth, Zigbee or WIMAX protocol, for example. In other embodiments, the communication device 840 includes a wired interface.

Optionally, the detection unit 830 may be communicatively coupled to a display 870. A representation of the information generated by the detection unit 830 can be displayed on the display 870. In some implementations, the display includes LEDs of different colors, e.g., a red LED and a green LED. The red LED can be activated when the predetermined characteristic is outside a normal range and the green LED can be activated if the predetermined characteristic is within the normal range. Alternatively or additionally, the display 870 may be capable of presenting a graphical or alphanumeric representation of the information.

After completing the assessment of the urine sample contained within the chamber 820, the urine can be expelled from the chamber 820 via a conduit 822. In some implementations, the conduit 822, e.g., tubing, can be configured to be routed through a gap between the toilet bowl 803 and the toilet seat 805. Alternatively, the conduit 822 can extend through an access port provided in the toilet bowl 803, which serves as a fluid pathway to return the expelled urine to the toilet bowl 803. In some embodiments, conduit 822 can share an access port with conduit 810. In some embodiments, conduit 819 and conduit 822 can use a separate access ports 807, 823. Although not shown, the conduit 822 can extend vertically upward from the access port 823 so that the distal end of the conduit 822 is above the water level within the toilet bowl 803. A seal is provided between the access port 823 of the toilet bowl 803 and the conduit 822 to prevent leakage of toilet water from the bowl 803 via the access port 823. In some embodiments, the conduit 822 can pass through the same access port 807 that accommodates conduit 810.

After completion of a urine assessment test, a cleansing operation can be performed. According to one cleansing approach, toilet water can be used to flush residual urine from the testing apparatus 800. A water supply line 852 can be connected to the water tank of the toilet 801 and transport fresh water to the capturing arrangement 802. An existing toilet and tank could be retrofitted by routing the water supply line 852 through a special flapper replacing a standard flapper. An irrigation manifold can be provided along the periphery of the capturing arrangement 802, which allows fresh water to cleanse the urine-receiving surface of the capturing arrangement 802. The fresh water received from the toilet tank passes through the diverter 806 and the conduits 808 and 810, thereby cleansing these structures. The fresh water passing through the conduit 810 fills up and passes through the chamber 820 of the external housing 818. The cleansing water passing through the testing apparatus 800 is expelled back into the toilet bowl 803 via conduit 822. A second water supply line 850 can be added to supply fresh water from the toilet tank directly to the chamber 822 to enhance cleansing of the chamber 822.

In some embodiments, a cleansing solution can be introduced into the cleansing operation at a convenient location. For example, a dispensing unit can be installed within the tank of the toilet 801 and connected to the water supply line 852. The dispensing unit can be configured to dispense a predetermined volume of cleaning solution (e.g. bleach, citric acid, detergent) into the water supply line 852 during each cleansing cycle. In other embodiments, a dispensing unit can be installed near or within the external housing 818 and fluidically connected to the water supply line 850. A predetermined volume of cleaning solution can be dispensed into the water supply line 850 and pumped into the irrigation manifold of the capturing arrangement 802 and, if desired, into the chamber 820 during each cleansing cycle.

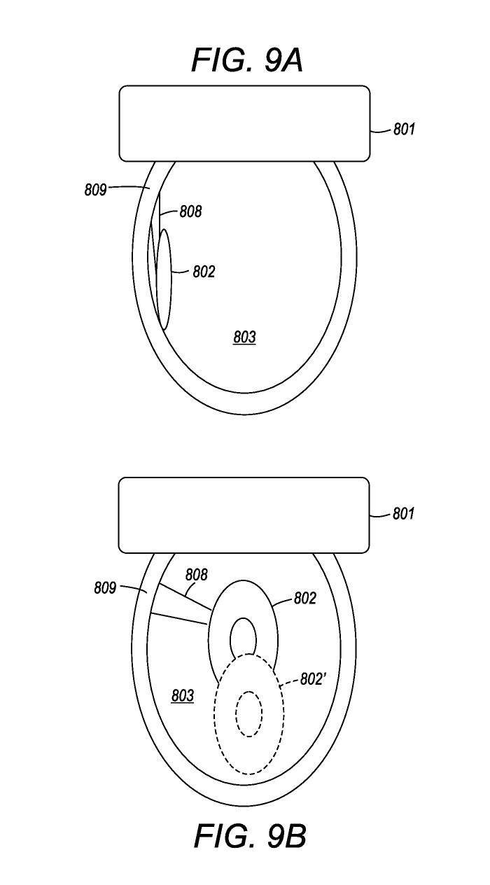

FIG. 9A is a top view of a toilet 801. In FIG. 9A, the capturing arrangement 802 is shown in a retracted configuration. In the retracted configuration, the capturing arrangement 802 and extension arm 808 are positioned at or near the peripheral rim 809 of the toilet bowl 803. The capturing arrangement 802 shown in the embodiment of FIG. 9A may be collapsible between a relatively circular shape and an elongated elliptical shape. The reduced profile of the capturing arrangement 802 when in the retracted configuration allows the toilet to be used in a normal manner (i.e., without urine capture and testing). In some embodiments, the capturing arrangement 802 and extension arm 808 are mounted to and deployed from a replaceable toilet seat. In other embodiments, the capturing arrangement 802 and extension arm 808 are mounted to and deployed from the peripheral rim 809 of the toilet bowl 803.

FIG. 9B shows the capturing arrangement 802 and extension arm 808 in a deployed configuration. In the deployed configuration, the capturing arrangement 802 is positioned at or near the center of the toiled bowl 803, and assumes a relatively circular shape. Depending on the gender of the person whose urine will be subject to testing, the capturing arrangement 802 can be positioned at an appropriate location within the toiled bowl 803 for receiving urine from females (802 in solid lines) and males (802′ in phantom). In some embodiments, the capturing arrangement 802 can be moved between retracted and deployed configurations using manual effort. In other embodiments, a motor can be used to move the capturing arrangement 802 between retracted and deployed configurations. Moving the capturing arrangement 802 to the deployed configuration can automatically activate the testing apparatus 800, such as by enabling power delivery to various electrical and electronic components (e.g., sensors, pumps, detection unit, and communication device) of the apparatus 800.

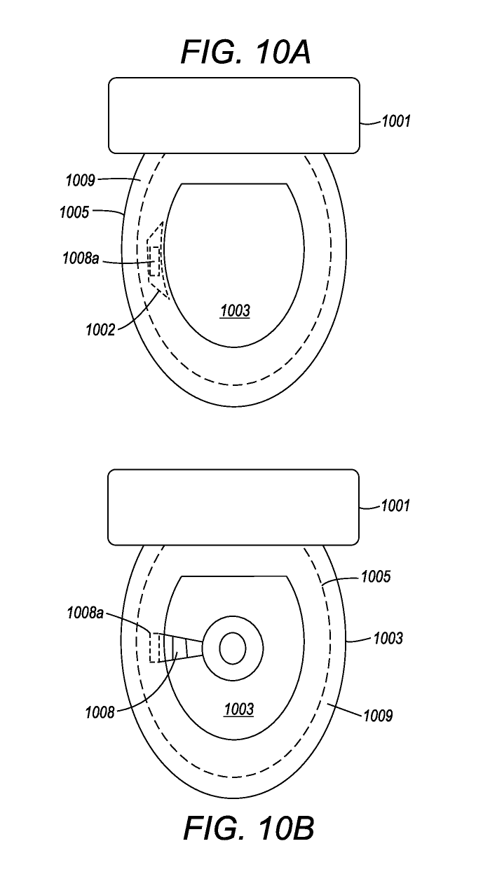

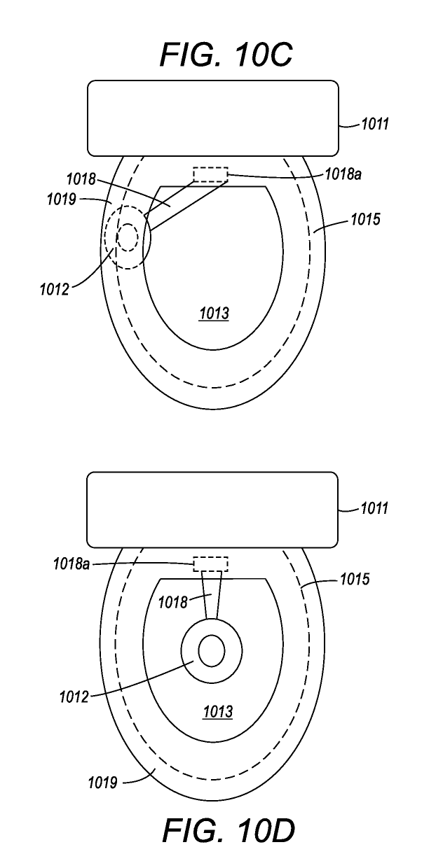

FIGS. 10A and 10B show a capturing arrangement comprising a shallow funnel 1002 that fits under the toilet seat 1005 and can be rotated into the bowl. In FIG. 10A, the capturing arrangement 1002 is shown in a retracted configuration. In the retracted configuration, the capturing arrangement 1002 and extension arm 1008, which may be a telescoping extension arm, are positioned at or near the peripheral rim 1009 of the toilet bowl 1003. In some embodiments, the mounting portion 1008a of extension arm 1008 is coupled to the toilet seat 1005. The capturing arrangement 1002 is mechanically coupled to the extension arm 1008 and can be deployed from a toilet seat 1005. In some other embodiments, the capturing arrangement 1002 and extension arm 1008 are mounted to and deployed from the peripheral rim 1009 of the toilet bowl 1003.

FIG. 10B shows the capturing arrangement 1002 and extension arm 1008 in a deployed configuration. During deployment of the capturing arrangement 1002, the capturing arrangement rotates with and extension arm 1008 rotate upward in a direction normal to the surface of the toilet seat 1005. In the deployed configuration, the capturing arrangement 1002 is shown positioned at or near the center of the toilet bowl 1003. The capturing arrangement 1002 can be positioned at any appropriate location within the toilet bowl 1003 for receiving the urine sample.

FIGS. 10C and 10D illustrate another configuration of a toilet seat that includes at least a portion of the testing apparatus. In the embodiment illustrated in FIGS. 10C and 10D, the capturing arrangement comprises a shallow funnel 1012 that fits between the toilet seat 1015 and the rim 1019 of the toilet bowl 1013. The capturing arrangement 1012 can be rotated into the bowl. In FIG. 10C, the capturing arrangement 1012 is shown in a retracted configuration and the mounting portion 1018a of extension arm 1018 is coupled to the toilet seat 1015 near the rear of the seat 1015, although the extension arm 1018 may be coupled at other locations. The capturing arrangement 1012 is mechanically coupled to the extension arm 1008 and can be deployed from a toilet seat 1015.

FIG. 10D shows the capturing arrangement 1012 and extension arm 1018 in a deployed configuration. During deployment of the capturing arrangement 1002, the capturing arrangement and extension arm 1018 rotate parallel to the surface of the toilet seat 1015. In the deployed configuration, the capturing arrangement 1012 is positioned at or near the center of the toilet bowl 1013 and/or at any appropriate location within the toilet bowl 1013 for receiving the urine sample.

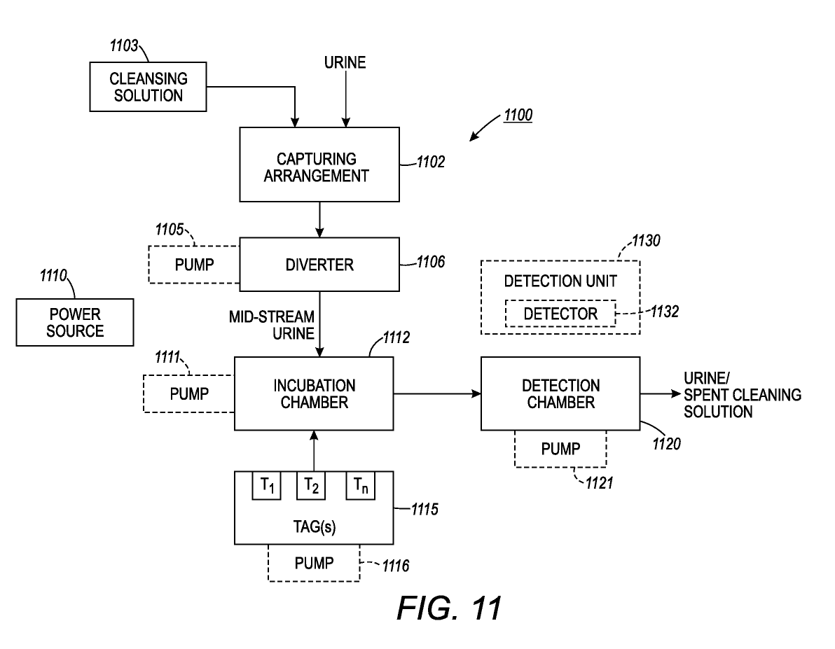

FIG. 11 illustrates a functional schematic of a testing apparatus 1100 in accordance with various embodiments. The testing apparatus 1100 includes a urine capturing arrangement 1102 fluidically coupled to a diverter 1106. The capturing arrangement 1102 is configured to receive urine, e.g., from a person using a toilet or other urine collection device. For example, in some embodiments, at least a portion of the testing apparatus may be affixed to the toilet seat of the toilet. The capturing arrangement 1102 is also fluidically coupled to a source of cleansing solution of 1103 in accordance with some embodiments. The diverter 1106 is fluidically coupled to an incubation chamber 1112. The diverter 1106 is configured to pass urine received by the capturing arrangement 1102 to the incubation chamber 1112. In some embodiments, a pump 1105 may be used to facilitate transport of the urine from the diverter 1106 to the incubation chamber 1112. A receptacle 1115 is configured to contain one or more specificity tags (T₁-Tₙ), such as one or more antibody—dye conjugates, selected for detecting one or more predetermined characteristics (e.g., analytes) of the urine sample. In some embodiments, one or more pumps 1116 or other dispensing mechanism(s) can be used to facilitate transport of the one or more tags contained in the receptacle 1115 to the incubation chamber 1112. The testing apparatus 1100 includes a power source 1110 that supplies power to the power-consuming components of the apparatus 1100 (e.g., pumps, sensors, detectors, valves, communication device, etc.). In some embodiments, the power source 1110 includes standard batteries. In other embodiments, AC power from the home or facility can be connected to the testing apparatus 1100 and serve as a source of power for the apparatus 1100.

The urine contained within the incubation chamber 1112 and the tags received from the receptacle 1115 are allowed to mix for a predetermined duration of time. After expiration of the predetermined duration of time, the mixture of urine and one or more tags is communicated to a detection chamber 1120. A metering sensor coupled to a processor (not shown) of the apparatus 1100 can be used to coordinate the transfer of urine through the various chambers and components of the apparatus 1100. For example, in some embodiments, the diverter 1106 is controlled by a liquid metering sensor. The desired portion of the urine flow is diverted to the incubation chamber 1112 when appropriate sensing conditions are met. For example after the initial 20 ml of urine have been omitted from the measurement, 15 ml of urine are diverted into the incubation chamber. Another approach could be to omit the first 10 seconds of the urine stream and divert the rest into the incubation chamber 1112. A metering sensor could be implemented by a liquid flow speed sensor, a thermometer, thermistor or other temperature sensor, a timer, an optical liquid plug detector or a combination of these metering sensors.

A detection unit 1130 is situated in proximity to the detection chamber 1120. A detector 1132 of the detection unit 1130 is configured to sense for presence of a predetermined characteristic or multiplicity of characteristics in the volume of the urine contained in the chamber 1120. After completion of the urine assessment by the detection unit 1130, the urine contained within the chamber 1120 is expelled, such as by use of a pump 1121.

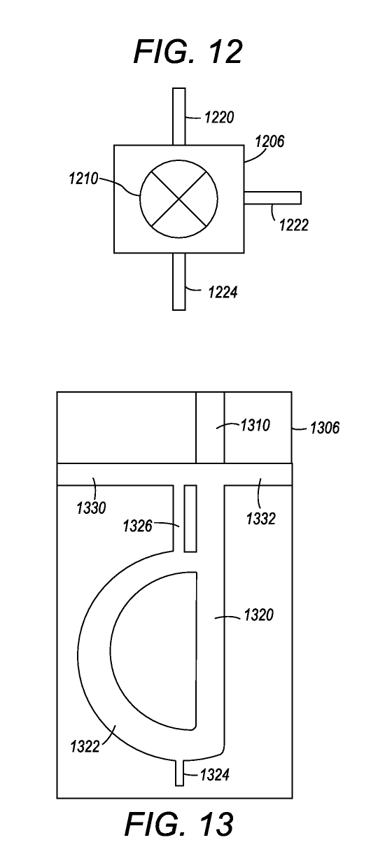

Referring to the diverter 1106, various implementations can be employed to pass urine to the detection chamber 1120 for assessment by the detection unit 1130, e.g., mid-stream urine. A mid-stream of urine is generally understood in the medical community to be one in which the first half of bladder urine is discarded and the last half or a portion thereof is collected for evaluation. The first half of the stream serves to flush contaminating cells and microbes from the outer urethra prior to capture. FIG. 12 shows an embodiment of a diverter 1206 configured to facilitate capture of bladder urine within the context of a testing apparatus described herein. The diverter 1206 includes a valve 1210 arranged to selectively prevent and enable passage of urine between a capturing arrangement (see, e.g., 402, 502, 602, 702, and 802 of FIGS. 4-8, respectively) and a chamber configured to collect the urine (see, e.g., 420, 520, 620, 720, and 820 of FIGS. 4-8, respectively).

The diverter 1206 includes an inlet port 1220 which is fluidically coupled to a capturing arrangement adapted for use at the toilet. The inlet port 1220 is fluidically coupled to a first port 1222 and a second port 1224 via the valve 1210. In a first position, the valve 1220 diverts urine passing through the inlet port 1220 into the first port 1222, and prevents passage of the urine into the second port 1224. In a second position, the valve 1210 diverts urine passing through the inlet port 1220 into the second port 1224, and prevents passage of the urine into the first port 1222. At the initiation of a urine testing cycle, the valve 1210 is moved to the first position, so that first-voided urine is transported through the first port 1222 and discarded, such as by being expelled into the toilet bowl. After a predetermined duration of time, the valve 1210 is moved to the second position, allowing urine passing through the inlet port 1220 pass through the second port 1224 and into a chamber that collects the urine for subsequent testing.

In some embodiments, the valve 1210 may be controlled by a metering sensor. The desired portion of the urine flow is diverted to the sensing section of the device when appropriate sensing conditions are met. For example after the initial 20 ml of urine have been omitted from the measurement, 15 ml of urine are diverted into the sensing section. Another approach could be to omit the first 10 seconds of the urine stream and divert the rest into the sensing section. A metering sensor could be implemented by a liquid flow speed sensor, a thermometer, thermistor or other temperature sensor, a timer, an optical liquid plug detector or a combination of these metering sensors. The metering sensor can be coupled to a processor of the testing apparatus. The control signal generated by the metering sensor causes the valve 1210 to move between the first and second positions described above. The predetermined duration is a measure of time from the beginning of urination to a time during urination in which a person's urine stream can be considered suitable for testing, such as is required by a standard urinalysis. For example, in some embodiments, the metering sensor is a timer that moves the valve after a predetermined duration. The predetermined duration can be established based on average urination data for a population of individuals or can be tailored for the individual using the testing apparatus. For example, an individual's total urination time can be measured on a repeated basis, and an average urination time can be calculated using the testing apparatus deployed at the individual's toilet. The calculated average urination time for the individual can be used to establish the predetermined duration (e.g., 50% of the individual's average urination time) of the timer.

FIG. 13 illustrates a diverter in accordance with other embodiments. The diverter 1306 shown in FIG. 13 includes an inlet port 1310, one or more outlet ports 1330, 1332, and a manifold 1320. The manifold 1320 is configured to divert first-voided urine received from the inlet port 1310 to a catch vessel 1322, which is shown as a curved tubular structure in FIG. 13. The catch vessel 1322 has a volume sufficient to hold first-voided urine that passes through the inlet port 1310. As urine is received through the inlet port 1310, the catch vessel 1322 continues to fill until the manifold 1320 is completely filled. Having contained the first-voided urine within the manifold 1320, subsequently received urine passing through the inlet port 1310 is diverted through the one or more outlet ports 1330, 1332, and constitutes urine that can be collected for testing. Upon completion of a urine testing cycle, the diverter 1306 can be flushed with a cleansing solution (e.g., with fresh water), with the first-voided urine being expelled via channels 1326, 1324, and outlet ports 1330, 1332. FIG. 13 depicts one embodiment of a volume controlled diverter. Other embodiments can utilize valving that is, for example, based on buoyancy valves that close off a disposal container after it is filled and divert additional urine volume into the collection channel for urine.

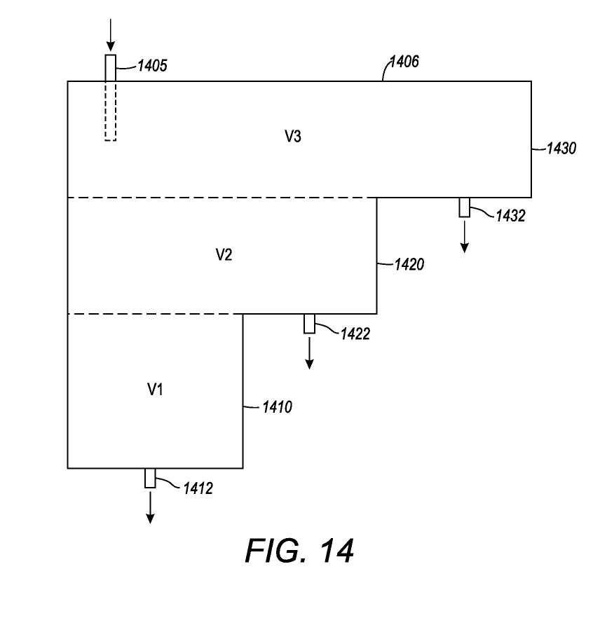

FIG. 14 illustrates a diverter in accordance with further embodiments. The diverter 1406 shown in FIG. 14 includes a tiered capture vessel structure, and inlet port 1405, and an outlet port 1412. In the embodiment shown in FIG. 14, the tiered capture vessel structure includes three sections 1410, 1420, and 1430, each having a respective outlet port 1412, 1422, and 1432. In accordance with some embodiments, a two-tiered capture vessel structure can be employed, thereby obviating the need for section 1430 and outlet port 1432. Urine received from a person using a toilet equipped with a testing apparatus of the present disclosure is directed through the inlet port 1405 and collects within a first section 1410 of the diverter 1406. The first section 1410 has a volume V1 that is sufficient to capture first-voided urine received from the inlet port 1405. The first-voided urine begins to fill the first section 1410 while at the same time begins to drain from the first section 1410 at a relatively slow rate through outlet port 1412. After filling the first section 1410 with first-voided urine, additional urine begins to fill the second section 1420. Because the first-voided urine drains from the outlet port 1412 concurrently with additional urine filling the second section 1420, urine collected within the second section 1420 and draining out of outlet port 1422 constitutes urine. Mixing of urine from V1 into V2 needs to be avoided for example by check valves or ensuring a laminar flow between V1 and V2. The volume V2 of the second section 1420 is selected to match that of the chamber to which the outlet port 1422 is fluidically connected. In some embodiments, a third section 1430 can be included to collect urine in excess of the desired volume of urine. For example, after filling the second section 1420 with urine, any additional urine beyond that contained within the second section 1420 can spill into the third section 1430 and drain through outlet port 1432. It will be understood that various diverter implementations are contemplated and that those described herein are provided for non-limiting illustrative purposes.

A testing apparatus of the present disclosure can be implemented to include one or more detection units configured to assess urine received from an individual. In some embodiments, a testing apparatus includes a detection unit configured to perform at an electrochemical assessment of a volume of urine. In other embodiments, a testing apparatus includes a detection unit configured to perform a chemical assessment of the volume of urine. In further embodiments, the testing apparatus includes a detection unit configured to perform a colorimetric assessment of a volume of urine. In some embodiments, a testing apparatus can be implemented to include a detection unit configured to perform a biochemical assessment of a volume of urine. According to further embodiments, a testing apparatus includes a detection unit configured to perform an immunoassay assessment of a volume of urine. It is understood that a testing apparatus can incorporate one or a multiplicity of these and other detection units.

In accordance with various embodiments, a testing apparatus deployable at a toilet includes a detection unit comprising an optical flow cytometer. According to some embodiments, an optical flow cytometer device is configured to detect the concentration of cells in urine in real-time to monitor the health of a kidney transplant, for example. According to some embodiments, an optical flow cytometer device is configured to detect the concentration of lymphocytes and/or RTCs in urine in real-time to monitor the health of a kidney transplant, for example. An optical flow cytometer can be deployed at a toilet and fluidically coupled to a capture apparatus within the toilet bowl (e.g., a funnel) for urine capture, thereby establishing a fluidic path to the cytometer. Deployment of the cytometer at the toilet allows real-time collected urine to be analyzed for the concentration of lymphocytes or RTCs, as well as for other analytes of interest. The flow cytometer can be part of the detection unit as described in FIGS. 4, 5, 6, 7, 8, 11. A testing apparatus that includes a flow cytometer can be deployed in clinics, patient homes or long-term care facilities where the patient's kidney transplant or other renal disorder can be monitored at least twice a day, for example, during regular urination. Aside from kidney transplant patients, the flow cytometer device installed at the toilet can be tailored to detect other analytes of interest in urine with wide applicability in diagnosis and therapeutic monitoring of disease states such as chronic kidney disease, diabetes, etc. For example, wireless communication capability can be incorporated into the device allowing for seamless communication between the patient and the kidney transplant team in the hospital to monitor the patient's urinary health.

Traditional urinalysis involves centrifuging the urine sample (˜12 ml) and re-suspending the sample in 250 μl of urine, which is analyzed on a slide under a microscope where observed elements are quantified as the number per high power field. Automated urinalysis instruments such as Sysmex UF-1000i, Iris iQ200, sediMAX greatly increase the throughput in a lab-based setting and dramatically reduce labor and turn-around-times for results. From a technological point of view, every automated urinalysis instrument uses a different technology to classify and quantify urine sediment particles and offers an improvement in standardization over manual microscopy by eliminating potential inter-technician variability during slide interpretation. The Sysmex UF-1000i is presently the only instrument available in the market which employs flow cytometry and fluorophores to categorize cells in uncentrifuged urine labeled with fluorophores according to their fluorescence, size, impedance, and forward scattered light. Even though the instrument features adequate sensitivities, its specificity is still poor for differentiating the different elements, which therefore must be confirmed by manual microscopy by a trained technician following the cytometry measurement. Since a trained technician is crucial for initial urine sample preparation and positive manual identification of cells in the urine when using the Sysmex UF-1000i, this precludes the use of this instrument in a home or point-of-care setting which poses a significant barrier for patient compliance. In addition, the Sysmex UF1000i is currently priced at $125,000, posing a significant barrier for adoption of urine cytology as a routine test for patients in an out-patient clinic setting at site of sample capture.

Embodiments of the disclosure provide a new way of performing urine screening for renal transplant patients, bladder cancer, (chronic) bladder infection patients, diabetics, and patients with other significant (renal) disorders. Various embodiments disclosed herein are based on selective cell counts in urine samples. According to some embodiments, a detection unit is configured to detect cells by native protein fluorescence (e.g., excited around 280 nm) and size/shape analysis of the detected particles. Some embodiments are directed to avoiding any kind of specificity reagent to achieve a low-cost monitoring tool and an unrestricted means to dispose of the unaltered sample in the regular waste stream. Minimal sample preparation prevents any complication in reproducibility, while the frequent and high sample throughput ensures sensitivity. The specificity of the monitoring tool could be provided by a number of orthogonal metrics, e.g., the intensity of native protein fluorescence, by cell size, concentration and absolute count, and by the long term development of these values. In particular, an increase in cell count, especially of lymphocytes, renal tubular cells, and polymorphonuclear cells which can be a significant early predictor of transplant rejection. Table 1 below provides representative mean cell values in urine samples in acute renal rejection cases, which can be quantified and monitored over time using a testing apparatus of the present disclosure.

TABLE 1

| | | | | Early vs. | |
|---|---|---|---|---|---|
| | | Pre- | | Pre- | Early vs. |
| Cell Type | Early | rejection | Rejection | rejection | Rejection |
| Renal tubular | 4.1 | 21.3 | 46.9 | <.01 | <.0001 |
| Macrophages | 5.45 | 11.7 | 19.7 | <.01 | <.02 |
| Polymorphonuclear | 10.8 | 57.5 | 89.8 | <.01 | <.07 |
| cells | | | | | |
| Lymphocytes | 5.9 | 15.2 | 34.6 | <.14 | <.01 |

Various embodiments are directed to in-home monitoring utilizing a fully automated device that performs urine analysis for at-risk individuals, such as renal transplant patients, after each urination. In a representative system, at least 2 ml urine sample is analyzed after each sampling, resulting in typically up to 40,000 detected cells per ml of urine. With an expected flow rate of 0.2 ml/min, the total analysis time is typically under 15 minutes. In some implementations, the detection area is limited to about 1×0.15 mm, and the channel thickness is about 50 μm. This detection area is well compatible with an approximate one-to-one image of a light emitting diode (LED) excitation source on the detection area. The size of the detection region and the anticipated throughput can result in a sample speed of about 1 m/s, a well suited speed for real-time particle evaluation. The result of the analysis can be displayed on a display communicatively coupled to the testing device and/or communicated to healthcare specialists that can assess an immanent risk of transplant rejection and advise appropriate steps such as a change in immunosuppressant titration.

A testing apparatus configured for deployment at the toilet provides for mid-steam urine sample capture and disposal, reagent-free urine analysis based on selective cell identification, and means to communicate these measurements according to various embodiments. As discussed previously, components of the testing apparatus can be integrated in a replacement toilet seat. In some embodiments, the analyzer (e.g., flow cytometer) of the testing apparatus requires minimal maintenance, ideally only automated cleaning with standard household cleaners, and simple battery replacement if necessary. Due to the use of relatively inexpensive LEDs, embodiments of the disclosure are scalable to a low-cost format while maintaining adequate sensitivity and specificity. Specifically for the kidney transplant patient population, a testing apparatus of the present disclosure can be retrofitted in a home toilet to perform daily routine urine cytology with high compliance, to monitor or quantify allograft rejection markers for early failure diagnosis or to titrate immunosuppressive medication dose.

In accordance with various embodiments, a detection unit can be configured to detect cells in a sample of urine by native protein fluorescence. Reference is made to Table 2 below, which provides excitation and emission data for various representative metabolites. When exciting at 280 nm, for example, native fluorescence of proteins (tryptophan, tyrosine) dominates the UV-fluorescence emission between 300 to 370 nm, while Riboflavin emits in the visible range. Riboflavin's fluorescence signature can be used to identify eosinophils. Visible NADH (nicotinamide adenine dinucleotide) fluorescence is not as effectively excited at 280 nm, wavelengths around 260 nm or 340 nm can be used to do so.

TABLE 2

| | | Fluorescence | Extiction | |
|---|---|---|---|---|
| | Excitation | emission | coefficient | Quantum |
| Molecule | (nm) | (nm) | (1/(cm M)) | yield |
| NAD+ | 260 | NA | 16000 | NA |
| NADH | 260 | 460 | 14000 | 0.019 |
| Riboflavin | 263 | 531 | 34845 | 0.3 |
| Tyrosine | 275 | 303 | 1404 | 0.13 |
| Tryptophan | 280 | 354 | 5500 | 0.12 |
| Phenylalanine | 257 | 280 | 191 | 0.022 |

Embodiments of a flow cytometer can be configured to implement spatial modulation detection in accordance with various embodiments. In spatially modulated detection, a continuously fluorescing bio-particle traverses an optical transmission pattern and thereby generates a time-dependent fluorescence signal. Correlating the detected signal with the known transmission pattern achieves high discrimination of the particle signal from background noise. It also allows for determining particle speed, particle size and particle aspect ratio. In conventional flow cytometry, the size of the excitation area is restricted approximately to the size of the particle. Spatial modulation detection according to the present disclosure uses a large excitation area which makes it possible to use LEDs or lamps as excitation light sources.

Traditional flow cytometry uses high excitation intensities in the detection area, while spatial modulation detection increase the total flux of fluorescence light that originates from a particle by integrating over a larger area. Therefore, it is possible to use inexpensive UV-LEDs that will soon be commercially available. The cost, power, and size constraints that a UV-laser would put on a system would be prohibitive to a deployment as a screening tool. According to one low-cost embodiment, for example, UV-LEDs with a total power of about 75 mW and a power density of 135 kW/m² can be used at a projected initial cost of less than about $400. This power density is sufficient compared to the current power density of 500 kW/m² that was used in the measurements determining leukocyte counts by native fluorescence excited at 266 nm.

A flow cytometer integrated in a urine testing apparatus of the present disclosure can be configured to determine particle size using spatial modulation detection. The use of spatial masks placed between the flow channel and detector provides several possibilities for size discrimination of continuously moving particles. One example of such a mask includes transparent regions at a fixed pitch of 30 μm. The actual opening widths decrease and then increase linearly by 1.5 μm. Maintaining a constant pitch is useful for frequency domain analysis to determine the particle speed and for particle triggering. The mask can have a folded design, with the larger openings at the edges and the smaller openings near the center to compensate for the excitation-intensity profile of the laser. A time-dependent signal arises from a fluorescing particle traversing this mask. The transmission times of particles passing under the openings is dependent on the widths of the opening. Approaches for determining the size of objects using the time-dependent signal are described in commonly owned U.S. patent application Ser. No. 14/181,530 entitled “Spatial Modulation of Light to Determine Object Length,” which is incorporated by reference in its entirety. Approaches for determining the size of color regions and and/or color homogeneity of objects using the time-dependent signal are described in commonly owned U.S. patent application Ser. No. 14/181,571 entitled “Determination of Color Characteristics of Objects Using Spatially Modulated Light,” which is incorporated by reference in its entirety.

Size measurements of particles can be used to gain cell specificity in urine samples. A detection window of cells can be gated to a size window of 9 to 20 μm, in order to exclude for example bacteria, cell clusters, and red blood cells from the relevant cell count. Such a detection window may allow for identification of renal tubular cells, macrophages, polymorphonuclear cells, and lymphocytes by size.

Cells of interest within a urine sample can be detected by autofluorescence according to some embodiments. Spatial modulation detection can be expanded from the visible into the ultra-violet spectral range and be used to detect objects, e.g., leukocytes, within a urine sample.

An experiment was performed to detect the presence of leukocytes in a buffer using a prototype flow cytometer in which cells were excited with a 20 mW, 266 nm CW laser at an intensity of about 500 kW/m². The cytometer detected the autofluorescence of the cells in the wavelength range of 280 nm to 380 nm. The particle speed in these measurements was tuned to about 0.8 m/s. In the experiment, a fluidic quartz channel and a periodic emission mask were used to detect and count the particles. The experiment verified that leukocytes can be counted in buffer solutions based on fluorescence intensity. Other urine constituents, for example red blood cells, bacteria, etc., can be excluded by fluorescence intensity. More effective gating can be achieved by utilizing size discrimination as described above.

Across a variety of technological areas, absorption-encoded micro beads can be designed and implemented to function as miniature, free flowing sensors. Analysis approaches described herein can involve detection of micro beads that have been encoded, e.g. filled, injected, coated, stained or treated, etc. with combinations of dyes having excitation or emission spectra that are distinguishable from one another. The k dyes can be used to encode n types of micro beads such that each type of micro bead includes the k dyes in a proportional relationship that is different from the proportional relationships of the k dyes included in others of the n types of encoded micro beads. Each of the n types of micro bead may have characteristics different from other types of the micro beads, e.g., size, shape, charge, porosity, surface characteristics, elasticity, material composition and/or each type of micro bead may be respectively functionalized to recognize particular analytes present in a urine sample.

For example, encoded micro beads can be added to a urine sample that is taken from person using a toilet equipped with a testing apparatus of a type previously described. The absorption encoded micro beads are detected by a detector (e.g., analyzer) configured to sense for one or more predetermined characteristics or properties of the urine sample based on information obtained from the micro beads. This information may be based on the presence of fluorescence intensity of a secondary binder (a so-called “sandwich assay”) that binds to the analyte of interest which in turn is bound to the primary binder on the surface of the bead. As another example, in some implementations, the encoded micro beads can be functionalized with recognition elements that interact with certain analytes in a urine sample. Encoded micro bead of a particular type have primary binders to a specific analyte functionalized to their surface while other types of micro beads have other types of binders encoded on their surface. During analysis of the urine sample, the types of micro beads present in the sample are detected based on the absorption spectra of the characteristic combination of dyes that identifies the type of micro bead. Additionally, information about the presence and/or quantity of one or more analytes in the urine sample can be determined based on whether and/or to what extent the analytes have interacted with the recognition elements of the micro beads.

Embodiments described herein involve the use of micro beads that can be deployed in a variety of applications, including analysis of system properties and/or detection of the presence and/or amount of an analyte in a urine sample. In some implementations, such as advanced diagnostics that are performed in a lab (e.g., see embodiments shown in FIGS. 3 and 7), multiple analytes in a urine sample may need to be detected in an assay. Encoded micro beads can be used in a multiplexed assay designed to identify the presence and/or amounts of multiple analytes.

In accordance with some embodiments, a detection unit for urinalysis can include a spatial filter having a plurality of mask features, and at least one optical detector positioned to sense light emanating from at least one object in the volume of urine moving along a flow direction with respect to the spatial filter. An intensity of the sensed light is time modulated according to the mask features. The optical detector is configured to generate a time varying electrical signal comprising a sequence of pulses in response to the sensed light. In some embodiments, the optical detector is configured to sense for native fluorescence emanating from the at least one object in the volume of the urine.

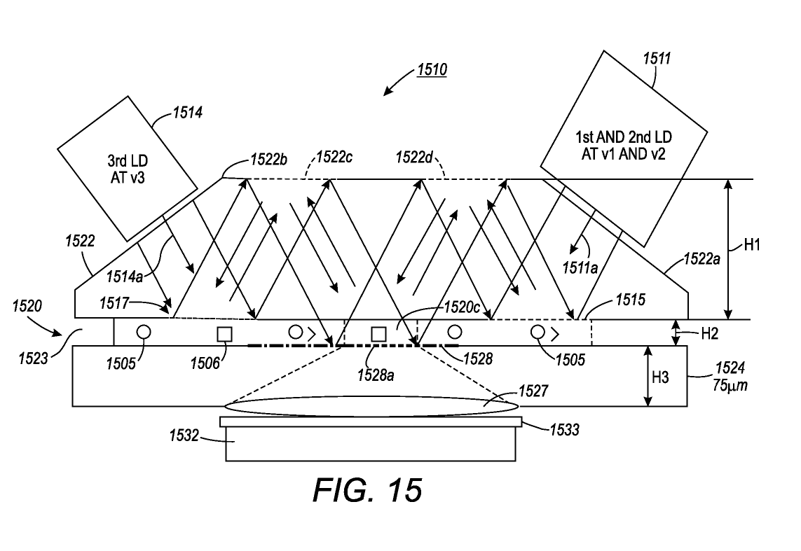

A representative detection unit is shown schematically in FIG. 15. The detection unit 1510 shown in FIG. 15 can be used for a compact flow cytometer that can perform single or multiple analyte detection performed on a urine sample acquired in real-time at a toilet. The detection unit 1510 includes a fluidic device 1520 which may be a fluidic chip. The fluidic device 1520 is adapted to receive the sample of interest to be tested (e.g., mid-stream urine), and to cause the sample to flow through a flow channel 1523 formed between confining members 1522, 1524. Gravity, a pump mechanism or other suitable device may be used to provide such sample flow. The urine sample may include cells 1505 of various types and micro beads 1506 of various types which have been encoded by k dyes, e.g., first and second dyes having first and second excitation characteristics. It is understood that a single type of micro bead can be used if detecting a single analyte in a urine sample is desired. A label antibody used to detect one or more analytes in a sample. Combined light source 1511, which provides combined first and second excitation light 1511a, is coupled to a first interface 1522a of the confining member 1522. A third light source 1514 generates third excitation light 1514a and is coupled to a second interface 1522b of the confining member 1522. The interfaces 1522a and 1522b are angled surfaces of the confining member 1522 to allow excitation light 1511a, 1514a from the light sources 1511, 1514 to propagate within the confining member 1522 and illuminate an excitation region 1520c of the flow channel 1523.

The combined light source 1511 emits combined excitation light 1511a that includes first excitation light and second excitation light. The first and second excitation light may be combined using collimating lenses and a beam splitter. First excitation light is centered at or peaks at a first wavelength λ1, and second light is centered at or peaks at a second wavelength λ2. A third light source 1514 may emit third excitation light 1514a that is centered at or peaks at a third wavelength λ3. The confining member 1522 is substantially transmissive to wavelengths λ1, λ2, and λ3. The first, second, and third light sources are preferably solid-state devices such as laser diodes or LEDs. One of them preferably emitting around 280 nm.

In the depicted embodiment, combined light 1511a is internally reflected by surface 1515 and then internally reflects against a first upper inner surface 1522d of confining member 1522 as shown in the figure before illuminating the excitation region 1520c of the flow channel. Reflection on surfaces 1522d and 1515 might be due to total internal reflection, partial reflection due to refractive index mismatches between 1522 and 1523 respectively between 1522 and its surrounding environment, or due to partially mirrored surfaces of 1522. Light 1514a is similarly internally reflected by a second lower mirror 1517 and then internally reflects against the second upper inner surface portion 1522c of confining member 1522 before illuminating substantially the same excitation region 1520a. In some cases, one or more of mirrors 1517, 1515 may be omitted and replaced with total internal reflection (TIR) at an air interface, e.g. by providing suitable air gaps (note that the flow channel 1520 can be redirected or reconfigured such that it does not reside in the vicinity of mirrors 1517, 1515).

The first excitation light, which is a first component of combined excitation light 1511a, is effective to excite light emission from the encoding dyes of the bead (while not substantially exciting light emission from the second or third fluorophores); the second excitation light which is a second component of combined excitation light 1511a is effective to excite light emission from the secondary binder (while not substantially exciting light emission from the first or third fluorophores); and the third excitation light 1514a is effective to excite light emission from native fluorophores in cells (while not substantially exciting light emission from the first or second fluorophores that encode the micro beads and detect the presence of secondary binders).

Light emanating from the various micro beads and cells 1505, 1506 is detected by photosensitive detector 1532. Detector 1532 may have an associated spatial filter 1528 in order to derive more information from the excited micro beads. Detector 1532 may have associated spectral filters (not shown) in order to separate the fluorescence of micro beads, cells and secondary binders. As illustrated in FIG. 15, the spatial filter 1528 can be disposed on the fluidic device or may be disposed in the path of light emitted by one or more of the light sources 1514, 1511and/or may be remotely imaged onto the flow channel. A working portion 1528a of the filter 1528, characterized by a sequence of transmissive and non-transmissive regions arranged along the longitudinal direction. Light that travels through the spatial filter is optionally imaged by an optional optical element 1527 such as one or more suitable lenses and/or mirrors onto the detector 1532. The optical element 1527 may provide magnification, in which case the detector area that receives light that traverses through the spatial filter 1528 may be larger than the working portion 1528a of the spatial filter.

The detector 1532 provides a detector output which varies in time in accordance with at least: the passage of excited micro beads through the detection portion(s) of the flow channel 1523; the pattern of transmissive and non-transmissive regions of the spatial filter 1528; and the modulation of the excitation light sources. The detector output may be evaluated and analyzed using various known signal analysis techniques. An optical emission filter 1533 may be provided for detector 1532 in order to block at least any residual excitation light that would otherwise fall on the detector 1532, while transmitting at least some of the light emission from the first, second, and third fluorophores.

In an exemplary embodiment, the detection unit 1510 may be made in a relatively small format suitable for use in POC applications, such as within a testing apparatus mounted near or on a toilet. In such embodiment, the dimensions H1, H2, and H3 in FIG. 15 may be as follows: H1 may be about 500 μm to about 4 mm; H2 may be about 25 to 100 μm; and H3 may be about 75 to about 300 μm, but these dimensions should not be construed to be limiting.

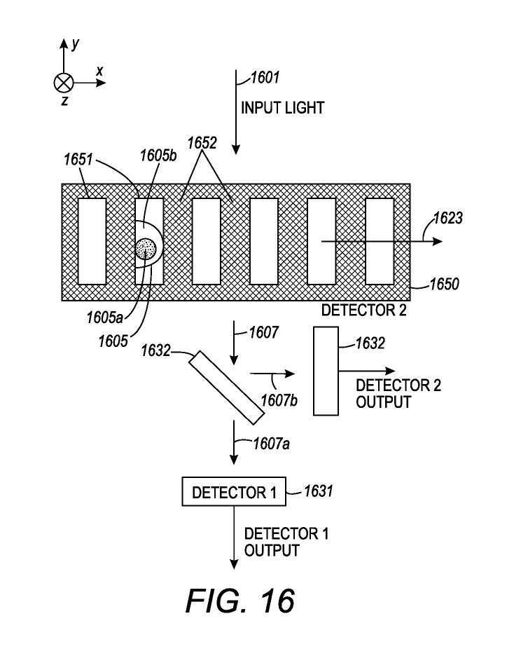

Another representative detection unit is shown schematically in FIG. 16. FIG. 16 conceptually illustrates a device configuration useful for performing urinalysis according to some approaches. As shown in FIG. 16, an object 1605 is moving along a flow path 1623 relative to spatial mask 1650 which includes substantially clear and opaque mask features 1651, 1652. Note that for illustration the spatial mask in FIG. 16 is shown oriented in the x-y plane but would actually be oriented so as to provide spatially modulated light to the detectors during use, e.g., the x-z plane. The object may be a single colored object or may be a multicolored object as depicted in FIG. 16. As the object 1605 moves along the flow path 1623, the input light 1601 causes the object to emanate light, e.g., due to scattering or fluorescence. Light may emanate from first and second portions 1605a, 1605b of a multicolor object 1605, causing regions 1605a, 1605b to emanate different optical spectra. The light 1607 emanating from object 1605 includes the light emanating from regions 1605a and 1605b. Regions 1605a and 1605b may be spatially discrete, partially overlapping or overlapping. The device illustrated in FIG. 16 includes a dichroic mirror 1632 that splits the emanating light 1607 into first and second components 1607a, 1607b having differing optical spectra. The spectrum of the first portion 1607a of light includes at least some of the wavelengths of the light from region 1605a. The spectrum of the second portion 1607b of light includes at least some of the wavelengths of the light from region 1605b.

A first detector 1631 is positioned to sense light 1607a and generates an electrical signal in response to the sensed light 1607a. A second detector 1632 is positioned to sense light 1607b and generates an electrical signal in response to the sensed light 1607b. Additional electronics, e.g., signal processor and/or analyzer (not shown in FIG. 16), can analyze the electrical signals generated by first 1631 and second detectors to 1632 to determine the color or colors of the object 1605.

In some embodiments, the number of spectral channels separated and detected by designated detectors may be two as described here. In other embodiments, the number of spectral channels may be larger than two. A series of dichroic mirrors could further split the emanating light into more spectral channels, sensed by designated detectors.

Various embodiments of the disclosure can be implemented to test for specific components and/or characteristics of a urine sample acquired in a manner discussed herein. According to some of the approaches discussed herein, counts of cells (or other particles) with native fluorescence that are present in the urine can be determined. These cells are particles are excited by the input light and in response emit a fluorescence that has a different wavelength range than the excitation light.

According to some of the approaches discussed herein, counts of cells (or other particles) with native fluorescence and other optical properties can be determined. The other optical properties can include colorimetric measurements of urine color, refractive index of the urine which may be used to determine specific gravity and/or absorption of proteins in the urine, e.g., at about 280 nm, to provide a proteinuria test.

According to some approaches discussed herein, analysis may be based on specificity tags can be added to the urine during testing. For example, in some implementations, the approaches may be used to detect the presence of or count of cells that bind to certain specificity tags and/or cells that express certain proteins that bind to specificity tags. In either implementation, detection of the tag allows the cell to be identified.

According to some approaches, analysis may include detecting the presence and/or concentration of various analytes present, e.g., free floating, in the urine. For this implementation, color encoded beads could be used. In one implementation these beads could provide specific primary binding sites for the analyte of interest on their surface. A fluorescently labeled secondary binder would then inform about the presence and quantity of the analyte of interest by the amount of fluorescence intensity from the secondary binder. This quantification method is often referred to as “sandwich assay”.

Optical testing of urine composition has the advantage of being “contact-free”. This reduces complications due to fouling or unwanted growth of biofilms and it allows for easy cleaning. A detection unit of a testing apparatus deployed at a toilet, for example, can be configured to sense for presence of one or more of a predetermined ion or trace metal, a predetermined protein or enzyme, a predetermined type of cell, a predetermined molecule, urine specific gravity, osmolality, pH, or a predetermined bacterium in a urine sample immediately following capture (e.g., at a toilet). Specific dissolved analytes could be detected by their characteristic absorption or autofluorescence. They could also be detected by (multiplexed) bead assays. A prominent example for such an assay is the commercially available platform technology xMAP® from Luminex.

For example, a detection unit of a testing apparatus deployed at a toilet can be configured to sense for presence of one or more of proteinuria, leukocytes, ketones, and glucose in a urine sample immediately following capture (e.g., at a toilet). The detector unit can be configured to perform one or more of a chemical, electrochemical, biochemical, colorimetric, or immunoassay assessment of a urine sample acquired in real-time at the location of capture.

Urinalysis performed by a testing apparatus disclosed herein can reveal diseases that have gone unnoticed because they do not produce striking signs or symptoms. Examples include diabetes mellitus, various forms of glomerulonephritis, and chronic urinary tract infections. Normal, fresh urine is pale to dark yellow or amber in color and clear. Normal urine volume is in the range of 750 to 2000 ml/24 hr. A testing apparatus deployed at a person's toilet can assess the color and volume of urine produced during a 24 hour period (and multiple days) to determine if the color and volume of urine falls within or outside of normal ranges. A red or red-brown (abnormal) color could be from a food dye, eating fresh beets, a drug, or the presence of either hemoglobin or myoglobin. If the sample contains many red blood cells, it will be cloudy as well as red. Turbidity or cloudiness of a urine sample may be assessed by the testing apparatus. Turbidity or cloudiness may be caused by excessive cellular material or protein in the urine. The detection unit could have the capability of measuring turbidity by backscattered light from the sample. Coloring of urine could be measured by providing multispectral (e.g. “white”) light within the detector and measurement of the absorption spectrum. This measurement could be simplified by measuring the light intensity of light transmitted through the urine by a multitude of intensity sensors, each one sensitive only to parts of the illumination spectrum for example by different filters.

The testing apparatus may be configured to test for pH of a urine sample, for example using standard pH electrodes. The glomerular filtrate of blood plasma is usually acidified by renal tubules and collecting ducts from a pH of 7.4 to about 6 in the final urine. However, depending on the acid-base status, urinary pH may range from as low as 4.5 to as high as 8.0. The change to the acid side of 7.4 is accomplished in the distal convoluted tubule and the collecting duct.

Similarly, measurements with ion-selective electrodes can determine the concentration of predetermined ions. Elevated potassium and sodium ions in conjunction with increased urine pH values have been shown to promote urinary bladder carcinogenesis.

The testing apparatus may be configured to test for specific gravity of a urine sample. Specific gravity, which is directly proportional to urine osmolality which measures solute concentration, measures urine density, or the ability of the kidney to concentrate or dilute the urine over that of plasma. Specific gravity of urine between 1.002 and 1.035 on a random sample is generally considered normal if kidney function is normal. Since the specific gravity of the glomerular filtrate in Bowman's space ranges from 1.007 to 1.010, any measurement below this range indicates hydration and any measurement above it indicates relative dehydration. If specific gravity is not >1.022 after a 12 hour period without food or water, renal concentrating ability is impaired and the person either has generalized renal impairment or nephrogenic diabetes insipidus. In end-stage renal disease, specific gravity tends to become 1.007 to 1.010. Any urine having a specific gravity over 1.035 is either contaminated, contains very high levels of glucose, or the person may have recently received high density radiopaque dyes intravenously for radiographic studies or low molecular weight dextran solutions. In such cases, 0.004 can be subtracted from the measurement for every 1% glucose to determine non-glucose solute concentration.

For routine clinical purposes urine specific gravity is measured by the refractive index (RI) of urine. The refractive index of urine could for example be measured with differential refractometer to compensate for temperature effects or with a refractometer based on critical angles between the sample and a refracting prism of known refractive index. Another implementation could be based on a Fabry-Pérot interferometer (etalon).

The advantage of such an implementation would be the potential for refractive index measurements and free protein absorption measurements (around 280 nm) in the same optical cavity. The term “optical cavity” refers herein to a light-transmissive region that is at least partially bounded by light-reflective components, with the light-reflective components and the light-transmissive region having characteristics such that a measurable portion of light within the light-transmissive region is reflected more than once across the light-transmissive region. An “optical cavity component” is a component that includes one or more optical cavities. To provide more specificity, several optical cavities could be included in the detection unit. Each chamber could for example be equipped with molecular weight cut-off membranes that exclude analytes with molecular weight exceeding the cut-off weight from the refractive index or the absorption measurement. With this method the contribution of different constituents to the RI could be determined for example serum albumin (67 kDa) compared to small molecules including creatinine (113 Da).

The testing apparatus may be configured to test for various proteins in a urine sample. Normally, only small plasma proteins filtered at the glomerulus are reabsorbed by the renal tubule. However, a small amount of filtered plasma proteins and protein secreted by the nephron (Tamm-Horsfall protein) can be found in normal urine. Normal total protein excretion does not usually exceed 150 mg/24 hours or 10 mg/100 ml in any single specimen. More than 150 mg/day is defined as proteinuria. Proteinuria greater than 3.5 gm/24 hours is considered severe and known as nephrotic syndrome. Various proteins can be detected and counted using methods discussed hereinabove.

One or more of glucose, ketones, nitrite, and leukocytes in a urine sample can be detected and quantified using a testing apparatus of the present disclosure. Less than 0.1% of glucose normally filtered by the glomerulus appears in urine (<130 mg/24 hr). Glycosuria (excess sugar in urine) generally indicates diabetes mellitus. Ketones (acetone, aceotacetic acid, beta-hydroxybutyric acid) resulting from either diabetic ketosis or some other form of calorie deprivation (starvation) can be detected using techniques described herein. Nitrite in a urine sample can be detected. A positive nitrite test indicates that bacteria may be present in significant numbers in urine. Leukocytes can be detected and quantified using techniques described herein. A positive leukocyte assessment results from the presence of white blood cells either as whole cells or as lysed cells.

Systems, devices, or methods disclosed herein may include one or more of the features, structures, methods, or combinations thereof described herein. For example, a device or method may be implemented to include one or more of the features and/or processes described herein. It is intended that such device or method need not include all of the features and/or processes described herein, but may be implemented to include selected features and/or processes that provide useful structures and/or functionality.

In the above detailed description, numeric values and ranges are provided for various aspects of the implementations described. These values and ranges are to be treated as examples only, and are not intended to limit the scope of the claims. For example, embodiments described in this disclosure can be practiced throughout the disclosed numerical ranges. In addition, a number of materials are identified as suitable for various implementations. These materials are to be treated as exemplary, and are not intended to limit the scope of the claims.

The foregoing description of various embodiments has been presented for the purposes of illustration and description and not limitation. The embodiments disclosed are not intended to be exhaustive or to limit the possible implementations to the embodiments disclosed. Many modifications and variations are possible in light of the above teaching.

What is claimed is:

1. A system, comprising:
a urine capturing arrangement configured to receive urine from a user of the toilet;
a chamber fluidically coupled to the capturing arrangement;
a diverter fluidically coupled between the capturing arrangement and the chamber, the diverter configured to divert a volume of the received urine to the chamber; and
a detection unit configured to sense optical properties of at least one object in the volume of the urine and to generate at least one electrical signal comprising information about the sensed object.

2. The system of claim 1, wherein the optical properties comprise native fluorescence of the at least one object.

3. The system of claim 1, wherein the detection unit is further configured to count a plurality of the objects in the volume of the received urine in the chamber.

4. The system of claim 3, wherein the detection unit is further configured to detect at least one of a size and a shape of the at least one object in the volume of the received urine in the chamber.

5. A system, comprising:
a urine capturing arrangement configured to receive urine from user;
a chamber fluidically coupled to the urine capturing arrangement;
a diverter fluidically coupled between the urine capturing arrangement and the chamber, the diverter configured to divert a volume of the received urine to the chamber; and
a detection unit configured to sense for presence of a predetermined characteristic in the volume of the urine and to generate at least one electrical signal comprising information about the predetermined characteristic.

6. The system of claim 5, wherein the volume of the received urine is a mid-stream volume.

7. The system of claim 5, wherein the diverter is controlled by a metering sensor, the diverter passing the received urine to the chamber only after elapsing of a predetermined time duration measured by the metering sensor.

8. The system of claim 5, wherein the diverter comprises a valve controllable between a first state and a second state, the valve diverting first-voided urine away from the chamber when in the first state and passing a mid-stream volume of the urine to the chamber when in the second state.

9. The system of claim 5, wherein the diverter comprises:
a manifold configured to divert first-voided urine away from the chamber and pass mid-stream volume of the urine to the chamber; or
a first port configured to receive first-voided urine, and a second port configured to receive a mid-stream volume of the urine.

10. The system of claim 5, wherein the urine capturing arrangement is coupled to a urine collection device, and the urine collection device comprises a toilet, a urinal or a bladder catheter.

11. The system of claim 5, wherein the urine capturing arrangement is mechanically coupled to a toilet and is movable relative to the toilet between a retracted configuration and a deployed configuration.

12. The system of claim 5, wherein one or more of the urine capturing arrangement, the chamber, the diverter, and the detection unit are mechanically coupled to a toilet seat.

13. The system of claim 5, wherein the detection unit is configured to sense for presence of a predetermined ion or trace metal, a predetermined protein or enzyme, a predetermined type of mammalian cell, a predetermined molecule, urine specific gravity, osmolality, pH, or a predetermined bacterium.

14. The system of claim 5, wherein the detection unit configured to sense for presence of at least one of proteinuria, leukocytes, ketones, and glucose.

15. The system of claim 5, wherein the detection unit is configured to sense for presence of at least one of lymphocytes, renal tubular cells, macrophages, and polymorphonuclear cells based on the at least one electrical signal.

16. The system of claim 5, wherein the detection unit is configured to perform one or more of an electrochemical assessment, a chemical assessment, a colorimetric assessment, a biochemical assessment, and an immunoassay assessment of the urine.

17. The system of claim 5, further comprising:
a display; and
a display controller configured to receive the at least one electrical signal from the detector and to control the display to provide a representation of the information on the display.

18. The system of claim 5, wherein the detection unit comprises an optical flow cytometer.

19. The system of claim 5, wherein the detection unit comprises:
a spatial filter having a plurality of mask features; and
at least one optical detector positioned to sense light emanating from at least one object in the volume of the urine moving along a flow direction with respect to the spatial filter, an intensity of the sensed light being time modulated according to the mask features, the detector configured to generate a time varying electrical signal comprising a sequence of pulses in response to the sensed light.

20. The system of claim 19, wherein the optical detector is configured to sense for native fluorescence emitted from the at least one object in the volume of the urine.

21. The system of claim 5, further comprising:
an incubation vessel fluidically coupled between the capturing arrangement and the chamber, the incubation vessel configured to receive the volume of the urine; and
at least one receptacle configured to store at least one specificity tag and to introduce the at least one specificity tag into the incubation vessel.

22. The system of claim 21, wherein the incubation vessel is configured to transfer the volume of the urine and the at least one specificity tag to the chamber after expiration of a predetermined incubation period.

23. The system of claim 21, wherein the detector is configured to sense for fluorescence emitted from the specificity tag attached to the at least one object in the volume of the urine.

24. The system of claim 5, wherein at least the capturing arrangement is adapted for coupling to a bowl or a seat of the toilet.

25. The system of claim 5, wherein:
at least the urine capturing arrangement is adapted for coupling to a urine collection device;
the chamber is detachable from the system while the volume of the urine is contained therein; and
the detection unit is configured for operation remotely of the toilet, the detection unit configured to sense for presence of the predetermined characteristic in the volume of the urine contained in the detachable chamber.

26. The system of claim 5, wherein:
the detection unit is configured for operation remotely of the toilet; and
further comprising tubing fluidically coupled between the chamber and the detection unit, the detection unit configured to sense for presence of the predetermined characteristic in the volume of the urine transported through the tubing from the chamber to the detection unit.

27. A method, comprising:
capturing a sample of urine within a chamber of a testing apparatus coupled to a urine collection device, the urine collection device comprising a toilet, urinal, or bladder catheter;
sensing, for presence of a predetermined characteristic in the volume of the urine within the chamber; and
generating at least one electrical signal comprising information about the predetermined characteristic.

28. The method of claim 27, wherein sensing comprises sensing for presence of at least one of lymphocytes, renal tubular cells, macrophages, and polymorphonuclear cells.

29. The method of claim 27, further comprising transmitting the at least one electrical signal to a remote location.

30. The method of claim 27, wherein sensing for presence of the predetermined characteristic in the volume of the urine within the chamber is performed using spatially modulated light.
